(19) United States
(12) Patent Application Publication
(10) Pub. No.: US 2018/0177786 A1
(43) Pub. Date: Jun. 28, 2018

(54) MONOAMINE OXIDASE INHIBITORS AND METHODS FOR TREATMENT AND DIAGNOSIS OF PROSTATE CANCER

(71) Applicant: University of Southern California, Los Angeles, CA (US)

(72) Inventor: Jean C. SHIH, Beverly Hills, CA (US)

(21) Appl. No.: 15/904,103

(22) Filed: Feb. 23, 2018

Related U.S. Application Data
(63) Continuation-in-part of application No. 15/688,497, filed on Aug. 28, 2017.
Application No. 15/688,497 is a division of application No. 14/615,416, filed on Feb. 5, 2015, now Pat. No. 9,675,620.
Application No. 14/615,416 is a continuation-in-part of application No. 13/559,431, filed on Jul. 26, 2012, now Pat. No. 9,771,625.
(60) Provisional application No. 61/511,920, filed on Jul. 26, 2011.
(60) Provisional application No. 61/937,425, filed on Feb. 7, 2014.

(51) Int. Cl. A61K 31/5375; A61K 31/357; A61K 45/06; A61K 31/381; A61K 31/366; A61K 31/405; A61K 31/15; A61K 49/16; A61K 31/138; A61K 31/4188; A61K 31/352; A61K 31/4045; C07D 209/24; A61K 31/137; C12N 15/113; A61K 31/135; A61K 31/495

(52) U.S. Cl. CPC A61K 31/5375; C12N 2310/531; A61K 2300/00; A61K 45/06; A61K 31/381; C12Y 104/03004; A61K 31/366; A61K 31/405; A61K 31/15; A61K 49/16; A61K 31/138; A61K 31/4188; A61K 31/352; A61K 31/4045; C07D 209/24; A61K 31/137; C12N 15/1137; A61K 31/135; A61K 31/495; C12N 2310/14; A61K 31/357

(57) ABSTRACT

A method of treating prostate cancer in a subject with biochemically recurrent prostate cancer is provided. The method includes administering to a subject in need thereof an effective amount of a pharmaceutical composition that includes phenelzine.

### CROSS-REFERENCE TO RELATED APPLICATIONS

This application is a Continuation-in-Part of U.S. application Ser. No. 15/688,497, filed on Aug. 28, 2017, which is a divisional of U.S. application Ser. No. 14/615,416, filed on Feb. 5, 2015, now U.S. Pat. No. 9,675,620, which is a Continuation-in-Part of U.S. application Ser. No. 13/559,431, filed on Jul. 26, 2012, now U.S. Pat. No. 9,771,625, which claims the benefit of priority of U.S. Provisional Application Ser. No. 61/511,920, filed on Jul. 26, 2011, all of which are incorporated herein by reference in their entirety. Also U.S. application Ser. No. 14/615,416 claims the benefit of priority to U.S. Provisional Application Ser. No. 61/937,425, filed on Feb. 7, 2014, which is a continuation of PCT/US15/14695 filed Feb. 5, 2015, which is a continuation of U.S. application Ser. No. 15/293,055, filed Oct. 13, 2016 the content of which is incorporated herein by reference in its entirety.

### FIELD OF THE INVENTION

The present invention relates generally to inhibition of monoamine oxidases (MAOs) and their inhibitors (MAOIs) as strategies to treat cancer, particularly prostate cancer. This invention also relates to imaging, screening, diagnostics, and therapeutic methods of cancer.

### BACKGROUND OF THE INVENTION

Prostate cancer is the third most common cause of death from cancer in men of all ages and is the most common cause of death from cancer in men over age of 75. Current treatments for prostate cancer include (1) hormonal therapy, (2) chemotherapy, (3) radiation therapy, and (4) surgery. However, they are only effective for patients during the early stages of the disease. There are also undesired side effects associated with each of these treatment modalities. Moreover, for patients with advanced stages of castration-resistant and metastatic prostate cancers, these treatments are only partially effective.

Supplemental or combination therapies may improve the outcome in advanced patients. For instance, patients subjected to androgen ablation therapy with either chemical castration with a LH-RH agonist or surgical castration have benefited by the combination with an antiandrogen like bicalutamide. Patients who failed these hormonal therapies are often benefited by selective chemotherapy such as docetaxel and denosumab and additional hormonal therapy to deplete residual endogenous androgen synthesis (e.g. a CYP17 inhibitor, abiraterone). Despite the improvement, these additional therapies, in general, are only capable of prolonged survival by a few months.

Prognosis and staging of prostate cancer are typically evaluated using the Gleason grading system. A Gleason score is given to prostate cancer based on its microscopic appearance. Cancers with a higher Gleason score are more aggressive and have a worse prognosis. A Gleason score is determined by a pathologist who visually inspects a biopsy sample and then assigning a score to the observed tumor pattern. However, the Gleason system is entirely reliant upon human visual examination, which is prone to error with significant limitations on early detection.

In view of the above, there is an urgent, unmet need for more effective mechanism-based therapies and noninvasive early-stage diagnostic techniques to differentiate indolent from virulent forms of prostate cancer so that overtreatment of this disease can be avoided.

### SUMMARY OF THE INVENTION

Briefly, the present invention is based, in part, on the surprising discovery that monoamine oxidases exhibit differential expressions/activities in cancerous cells and that inhibitors of monoamine oxidases (MAOs) are capable of repressing the growth of cancer cells in vitro and tumor xenografts in vivo.

One aspect of the present invention is directed to a method of treating prostate cancer in a subject with biochemically recurrent prostate cancer. The method includes administering to the subject in need thereof an effective amount of a pharmaceutical composition that includes phenelzine.

In one embodiment, the pharmaceutical composition is administered orally.

In another embodiment, the pharmaceutical composition is administered orally twice a day and the amount of phenelzine in each dose is about 30 mg.

In another embodiment, the pharmaceutical composition further includes an excipient.

In another embodiment, the pharmaceutical composition further includes a physiologically acceptable carrier.

In another embodiment, a primary endpoint is a proportion of patients who achieve a PSA decline of ≥50% from baseline.

In another embodiment, the method further includes one or more monoamine oxidase A (MAO-A) inhibitors.

In another embodiment, the method further includes a Chinese herb medicine and/or a plant extract with the one or more MAO-A inhibitors.

Examples of MAO-A inhibitors include but are not limited to the following:

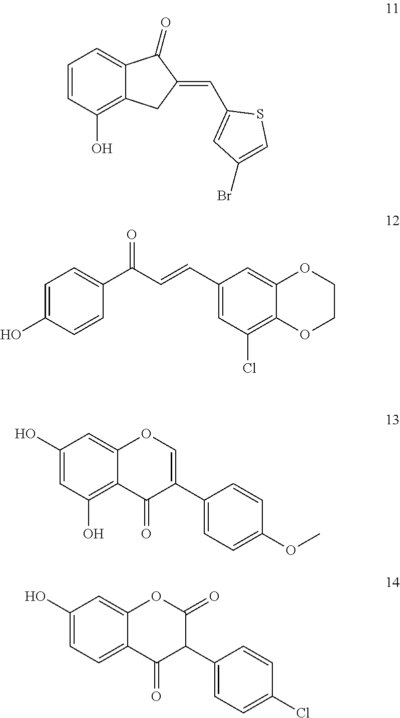 [SMILES: O=C1/C(=C/c2cc(Br)cs2)Cc2c(O)cccc21 ; O=C(/C=C/c1cc(Cl)c2c(c1)OCCO2)c1ccc(O)cc1 ; COc1ccc(-c2coc3cc(O)cc(O)c3c2=O)cc1 ; O=C1Oc2cc(O)ccc2C(=O)C1c1ccc(Cl)cc1]

Another aspect of the present invention is directed to a method of treating prostate cancer in a subject with a post-prostatectomy PSA ≥0.4 ng/ml or a post-radiation therapy PSA ≥2 ng/ml above a post-therapy nadir. The method includes administering to the subject in need thereof an effective amount of a pharmaceutical composition that includes phenelzine.

In one embodiment, the pharmaceutical composition is administered orally.

In another embodiment, the pharmaceutical composition is administered orally twice a day and the amount of phenelzine in each dose is about 30 mg.

In another embodiment, the pharmaceutical composition further includes an excipient.

In another embodiment, the pharmaceutical composition further includes a physiologically acceptable carrier.

In another embodiment, a primary endpoint is a proportion of patients who achieve a PSA decline of ≥50% from baseline.

In addition, MHI may be conjugated to phenelzine by any suitable chemical means known in the art.

Where prostate cancer is concerned, it is a further discovery of the present invention that MAO-A is, associated with chemo and radiation resistance in human prostate cancer whereas MAO-B has a unique expression pattern in human prostate cancer-associated stromal cells. As mentioned above, MAOs are mitochondrial-bound enzymes that catalyze the degradation of monoamine neurotransmitters and dietary amines via oxidative deamination. They are encoded by their genes located in the X chromosome [1, 2]. The by-product of MAO catalysis is hydrogen peroxide, a major source of reactive oxygen species (ROS), which can predispose cancer cells to DNA damage and promote tumor initiation and progression [3]. Modulation of intracellular ROS levels in prostate cancer cells could affect the sensitivity of prostate cancer cells toward hormonal, chemo- and radiation therapy [4]. Moreover, MAOs are responsible for the generation of ROS, in the presence of their biogenic amine substrates from the diet or physiological sources in an epithelial versus stromal cellular compartments. In addition, since prostate stroma is known to drive the progression of prostate cancer, by differentiating the forms, the amount, and the physical location of MAOs in prostate cancer tissue specimens, indolent forms of human prostate cancer may be differentiated from virulent forms. In short, it is an unexpected discovery of the present invention that MAOs are capable of serving as biomarkers for screening, diagnosing, and differentiating prostate cancer forms in patients. Based on the observation that MAO-A and MAO-B differs in their localization, a treatment strategy targeting both MAO-A in prostate cancer epithelium and MAO-B in prostate cancer-associated stroma is also devised.

Another aspect of the present invention is directed to a method of differentiating different forms of prostate cancer, comprising assaying MAO activity and location patterns in prostate tissues; and determining a cancer form characterization according to said MAO activity and location patterns. The said MAO activity can be determined, for example, by real-time PCR that measures the MAO-A expression in prostate biopsy.

Those skilled in the art will recognize that the above example is for illustration only and other currently known or future invented methods of measurement may also be used to determine MAO activity.

Another aspect of the present invention is directed to a method of screening a patient for risk of cancer, comprising assaying MAO activity in the patient; comparing said activity to a reference; and determining a risk level based on the comparison. The said MAO activity can be determined, for example, by real-time PCR that measures the MAO-A expression in prostate biopsy. The biopsy samples should be homogenized in Trizol, and RNA isolated. Next, 1 ug of total RNA will be reverse transcribed in 25 ul volume, then 2 ul of the sample (cDNA) is diluted 1/10 into 20 ul, 5 ul of this sample will be used as template for MAO A measurement. Another 2 ul will be diluted 1/50 into 100 ul, 5 ul of this sample is used for ribosomal RNA control template.

Another aspect of the present invention is directed to a method of monitoring treatment progress in a cancer patient being treated with a pharmaceutical composition comprising a NIR dye-based nano-conjugate. Methods in accordance with this aspect of the invention will generally include the steps of obtaining successive NIR image of the patient; and comparing said successive NIR images to determine progression of said treatment. The effect of conjugate on prostate tumor growth and metastasis can be determined by imaging and IHC analysis. The said imaging can be done, for example with Xenogen IVIS 200 instrument. This system allows researchers to use real-time, non-invasive imaging to monitor and record cellullar and genetic activity in vivo. Integrated into the system are both a bioluminescence system and a fluorescence system and the capability to easily switch between modalities. A laser scanner also provides 3D surface topography for single-view diffuse tomographic reconstructions of internal sources. Background noise is minimized while sensitivity is maximized using a 26 mm square CCD which is cryogenically cooled. Scans generally take 1-10 minutes to complete with five field of view options ranging from 4 cms to 25 cms.

Another aspect of the present invention is directed to a method of modulating ROS levels in cells. Methods in accordance with this aspect of the invention will generally include the steps of contacting a cell with a MAO inhibitory agent. Suitable MAO inhibitory agent may be any of the MAO inhibitors, nano-conjugates, or pharmaceutical compositions described above. These agents can be used either alone or in combination with mitochondria-directed antioxidants, such as lipoic acid, N-acetyl-L-carnitine and N-Acetyl-L-cysteine.

Another aspect of the present invention is directed to pharmaceutical composition useful for treating prostate cancer. The composition includes MHI-phenelzine and a physiologically acceptable carrier.

Another aspect of the present invention is directed to a method of delivering phenelzine to a cancer cell. The method includes conjugating phenelzine to MHI and contacting said MHI-phenelzine to the cancer cell.

Another aspect of the present invention is directed to a method of inhibiting monoamine oxidase or reducing reactive oxygen species in a cell. The method includes contacting the cell with MHI-phenelzine and a physiologically acceptable carrier.

Another aspect of the present invention is directed to a method of treating prostate cancer in a subject in need of the treatment. The method includes administering MHI-phenelzine to the subject, wherein said MHI-phenelzine is capable of inhibiting or down-regulating monoamine oxidase in the cancer cells of the subject.

Other aspects and advantages of the present invention will become apparent from the following detailed description and the appended claims.

### BRIEF DESCRIPTION OF THE DRAWINGS

FIG. 1 (A-F). shMAOA knockdown (KD) of MAOA in human LNCaP (A-C) and C4-2 (D-F) PCa cells significantly reduced tumor xenograft growth (A, D), tumor weight (B, E) and tumor incidence (C, F) in athymic nude mice (subcutaneous injection, 6 mice per cell line, 3 tumor inoculation sites per mouse). shCon and shMAOA, WT and MAOA-KD cells. *,p<0.05, **,p<0.01.

FIG. 1 (G-I). shMAOA KD of MAOA in murine MCP3 (Pten/p53 −/−) PCa cells eliminated tumor xenograft growth in B6 immune-intact mice (subcutaneous injection, 6 mice per cell line. 3 tumor inoculation sites per mouse).

FIG. 2. PSA trend over time in a BCRPC patient treated with phenelzine. The data show a 74% decrease maintained for over 1 year.

FIG. 3. Waterfall plot of PSA decline following phenelzine treatment in patients with recurring prostate cancer.

### DETAILED DESCRIPTION

### Definition

Unless otherwise indicated herein, all terms used herein have the meanings that the terms would have to those skilled in the art of the present invention. Practitioners are particularly directed to current textbooks for definitions and terms of the art. It is to be understood, however, that this invention is not limited to the particular methodology, protocols, and reagents described, as these may vary.

“Treatment” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented.

A “therapeutically effective amount” of a monoamine inhibitor is an amount sufficient to carry out a specifically stated purpose. An “effective amount” may be determined empirically and in a routine manner in relation to the stated purpose.

A “Carrier” or “Carriers” as used herein include pharmaceutically acceptable, carriers, excipients, or stabilizers which are nontoxic to the cell or mammal being exposed thereto at the dosages and concentrations employed. The physiologically acceptable carrier may be a sterile aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants.

The present invention is directed to a method of treating prostate cancer in a subject with biochemically recurrent prostate cancer. The method includes administering to the subject in need thereof an effective amount of a pharmaceutical composition that includes phenelzine.

In addition, the present invention is directed to a method of treating prostate cancer in a subject with a post-prostatectomy PSA ≥0.4 ng/ml or a post-radiation therapy PSA ≥2 ng/ml above a post-therapy nadir. The method includes administering to the subject in need thereof an effective amount of a pharmaceutical composition that includes phenelzine.

In a preferred embodiment, the pharmaceutical composition is administered orally.

In another embodiment, the pharmaceutical composition is administered orally twice a day and the amount of phenelzine in each dose is about 30 mg.

In another embodiment, the pharmaceutical composition further includes an excipient.

In another embodiment, the pharmaceutical composition further includes a physiologically acceptable carrier.

In another embodiment, a primary endpoint is a proportion of patients who achieve a PSA decline of ≥50% from baseline.

In another embodiment, the method further includes a monoamine oxidase A (MAO-A) inhibitor or a salt thereof.

In another embodiment, the method further includes a Chinese herb medicine and/or a plant extract with the MAO-A inhibitor or a salt thereof.

Another aspect of the present invention is directed to a method of treating prostate cancer in a subject with a post-prostatectomy PSA ≥0.4 ng/ml or a post-radiation therapy PSA ≥2 ng/ml above a post-therapy nadir. The method includes administering to the subject in need thereof an effective amount of a pharmaceutical composition that includes phenelzine.

The following examples are provided in order to demonstrate and further illustrate certain embodiments and aspects of the present invention and are not to be construed as limiting the scope thereof. While such examples are typical of those that might be used, other procedures known to those skilled in the art may alternatively be utilized. Indeed, those of ordinary skill in the art can readily envision and produce further embodiments, based on the teachings herein without undue experimentation.

### Knock Down MAO A Specifically Reduces the Tumor Growth

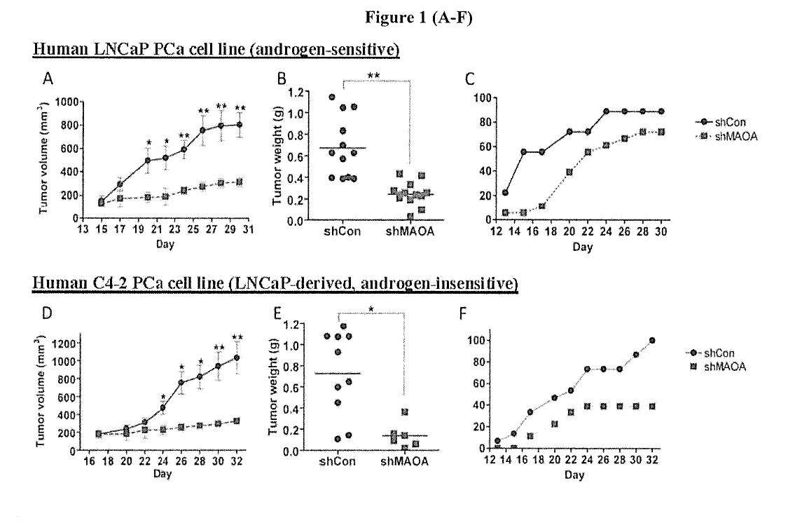

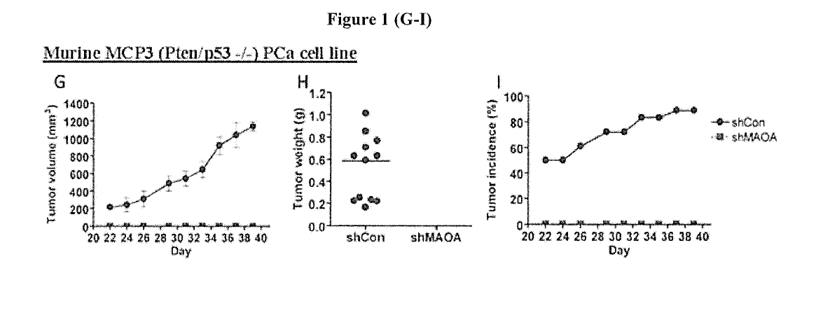

Data demonstrates that MAO A inhibitors reduce prostate cancer progression and metastasis. The following experiments demonstrate that this effect is due to inhibiting MAO A, not LSD1, (MAO A inhibitors also inhibits enzyme LSD1). In these experiments, the inventor specifically targets and silences tumor MAOA by lentiviral shMAOA. The results demonstrate significant inhibition of the growth of human PCa cells (both androgen-sensitive and androgen-insensitive) in vitro and tumor xenograft in vivo (FIG. 1A-F), further, it eliminates the growth of a murine tumor xenograft in immune-intact mice (FIG. 1G-I).

### MAO Inhibitor (Phenelzine) Reduces PSA Level in Prostate Cancer Patients

Based on data supporting therapeutic potential for targeting of MAOA in prostate cancer, a phase 2 clinical trial was initiated to explore the effect of phenelzine (Nardil®, a MAOA/B inhibitor) in patients with biochemically recurrent prostate cancer (BCRPC) (ClinicalTrials.gov Identifier: NCT02217709).

Eligibility included patients with elevated PSA after primary therapy defined by: post-prostatectomy PSA ≥0.4 ng/ml or post-radiation therapy PSA ≥2 ng/ml above a post-therapy nadir, non-castrate levels of circulating testosterone (>50 g/dl), and no evidence of metastatic cancer on standard imaging studies.

Patients were enrolled in an open-label fashion to receive a target dose of phenelzine 30 mg orally twice daily. The primary endpoint is the proportion of patients who achieve a PSA decline of ≥50% from baseline.

A preliminary analysis was performed after twelve evaluable patients were enrolled according to the Simon minimax two-stage design to define the probability of response to phenelzine (P1) as ≥20% and reject the drug if the response probability (P0) is ≤5% with power set at 0.8.

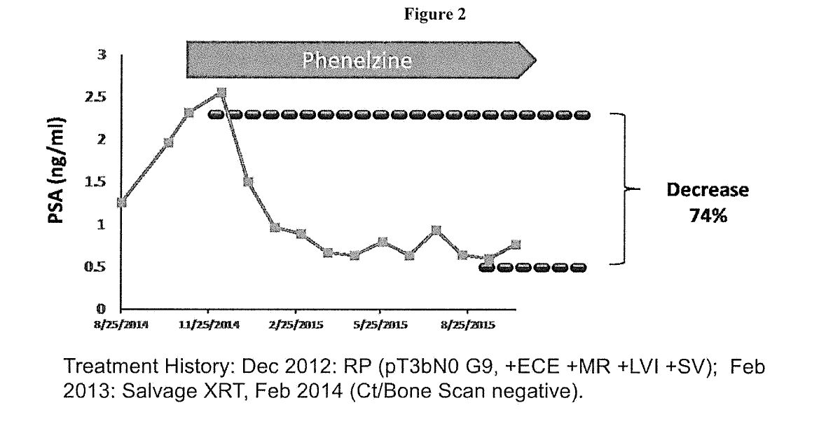

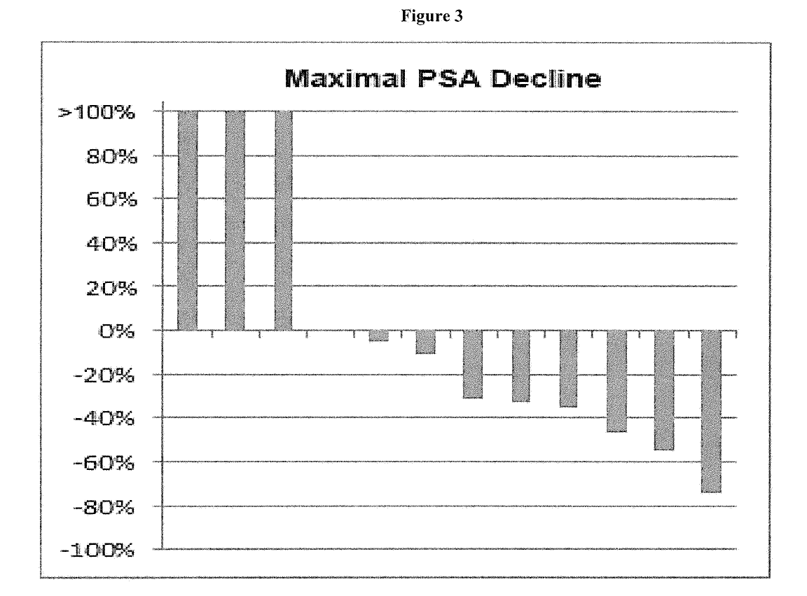

At the interim analysis, two subjects demonstrated ≥50% maximum decline in PSA level. Maximal PSA declines varying between 1-44% have been observed in six other subjects in the non-castrate group (see FIGS. 2 and 3). The adverse events profile varied greatly across subjects with some subjects reporting no ongoing toxicities while mild toxicities typically associated with MAOAIs were observed in others. Common toxicities reported included fatigue, dizziness, edema, and hypertension which were generally grade 1. The preliminary finding of ≥50% PSA decrease in 2/12=17% meets pre-specified criteria for continued enrollment towards the goal of 21 evaluable subjects.

Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the following claims.

### REFERENCES

The following references are incorporated herein by reference:
1. Bortolato, M., Chen, K., and Shih, J. C. (2008) Advanced Drug Delivery Reviews 60, 1527-1533
2. Shih, J. C., Chen, K., and Ridd, M. J. (1999) Annual Review of Neuroscience 22, 197-217
3. Josson, S., Nomura, T., Lin, J. T., Huang, W. C., Wu, D. Q., Zhau, H. E., Zayzafoon, M., Weizmann, M. N., Gururajan, M., and Chung, L. W. K. (2011) Cancer Research 71, 2600-2610
4. Trachootham, D., Alexandre, J., and Huang, P. (2009) Nature Reviews Drug Discovery 8, 579-591
5. Peehl, D. M., Coram, M., Khine, H., Reese, S., Nolley, R., and Zhao, H. J. (2008) Journal of Urology 180, 2206-2211
6. Zhao, H. J., Flamand, V., and Peehl, D. M. (2009) Bmc Medical Genomics 2, —
7. Flamand, V., Zhao, H. J., and Peehl, D. M. (2010) Journal of Cancer Research and Clinical Oncology 136, 1761-1771
8. True, L., Coleman, I., Hawley, S., Huang, C. Y., Gifford, D., Coleman, R., Beer, T. M., Gelmann, E., Datta, M., Mostaghel, E., Knudsen, B., Lange, P., Vessella, R., Lin, D., Hood, L., and Nelson, P. S. (2006) Proceedings of the National Academy of Sciences of the United States of America 103, 10991-10996
9. De Colibus, L., Li, M., Binda, C., Lustig, A., Edmondson, D. E., and Mattevi, A. (2005) Proceedings of the National Academy of Sciences of the United States of America 102, 12684-12689
10. Ueno, Y., Jose, J., Loudet, A., Perez-Bolivar, C., Anzenbacher, P., Jr., and Burgess, K. (2011) J Am Chem Soc 133, 51-55
11. Barth, B. M., Sharma, R., Altinoglu, E. I., Morgan, T. T., Shanmugavelandy, S. S., Kaiser, J. M., McGovern, C., Matters, G. L., Smith, J. P., Kester, M., and Adair, J. H. (2010) Acs Nano 4, 1279-1287
12. Altinoglu, E. I., Russin, T. J., Kaiser, J. M., Barth, B. M., Eklund, P. C., Kester, M., and Adair, J. H. (2008) Acs Nano 2, 2075-2084
13. Jose, J., Loudet, A., Ueno, Y., Wu, L., Chen, H. Y., Son, D. H., Barhoumi, R., Burghardt, R., and Burgess, K. (2011) Org Biomol Chem 9, 3871-3877

What is claimed is:

1. A method of treating prostate cancer in a subject with biochemically recurrent prostate cancer comprising: administering to the subject in need thereof an effective amount of a pharmaceutical composition comprising phenelzine.

2. The method of claim 1, wherein the pharmaceutical composition is administered orally.

3. The method of claim 2, wherein the pharmaceutical composition is administered orally twice a day and the amount of phenelzine in each dose is about 30 mg.

4. The method of claim 1, wherein the pharmaceutical composition further comprises an excipient.

5. The method of claim 1, wherein the pharmaceutical composition further comprises a physiologically acceptable carrier.

6. The method of claim 1, wherein a primary endpoint is a proportion of patients who achieve a PSA decline of ≥50% from baseline.

7. A method of treating prostate cancer in a subject with a post-prostatectomy PSA ≥0.4 ng/ml or a post-radiation therapy PSA ≥2 ng/ml above a post-therapy nadir comprising: administering to the subject in need thereof an effective amount of a pharmaceutical composition comprising phenelzine.

8. The method of claim 7, wherein the pharmaceutical composition is administered orally.

9. The method of claim 8, wherein the pharmaceutical composition is administered orally twice a day and the amount of phenelzine in each dose is about 30 mg.

10. The method of claim 7, wherein the pharmaceutical composition further comprises an excipient.

11. The method of claim 7, wherein the pharmaceutical composition further comprises a physiologically acceptable carrier.

12. The method of claim 7, wherein a primary endpoint is a proportion of patients who achieve a PSA decline of ≥50% from baseline.

13. The method of claim 1, further comprising one or more monoamine oxidase A (MAO-A) inhibitors.

14. The method of claim 13, further comprising a Chinese herb medicine and/or a plant extract.

15. The method of claim 7, further comprising one or more monoamine oxidase A (MAO-A) inhibitors.

16. The method of claim 15, further comprising a Chinese herb medicine and/or a plant extract.
